(19) United States
(12) Patent Application Publication
(10) Pub. No.: US 2026/0305970 A1
(43) Pub. Date: Oct. 8, 2026

(54) LUMBAR-VERTEBRA MAGNETIC RESONANCE IMAGING METHOD AND APPARATUS, COMPUTER DEVICE, READABLE STORAGE MEDIUM, AND PROGRAM PRODUCT

(71) Applicant: Hainan General Hospital, Haikou (CN)

(72) Inventors: Yihao GUO, Haikou (CN); Xiaoli XIONG, Haikou (CN); Feng CHEN, Haikou (CN)

(21) Appl. No.: 19/239,658

(22) Filed: Jun. 16, 2025

(30) Foreign Application Priority Data
Apr. 2, 2025 (CN) 202510412354.5

(51) Int. Cl. A61B 5/055; A61B 5/00; G01R 33/48

(52) U.S. Cl. CPC A61B 5/055; A61B 5/4566; G01R 33/4828

(57) ABSTRACT

The present disclosure relates to a lumbar-vertebra magnetic resonance imaging method and apparatus, a computer device, a computer-readable storage medium, and a computer program product. The method includes: acquiring a sequence for lumbar vertebrae in a magnetic resonance imaging process; the sequence including water-fat in-phase echo signals corresponding to different echo times; determining an initial transverse relaxation rate map corresponding to the lumbar vertebrae and an initial field map representing inhomogeneity of a static magnetic field applied to the lumbar vertebrae according to a signal intensity of each water-fat in-phase echo signal, an echo time of each water-fat in-phase echo signal, and a pre-constructed signal model; taking the initial transverse relaxation rate map and the initial field map as initialization reconstruction parameters of water-fat separation, and reconstructing a water-fat separation image of the lumbar vertebrae.

### CROSS REFERENCE TO RELATED APPLICATIONS

This application claims priority under 35 U.S.C. § 119 to Chinese Patent Application No. 2025104123545, filed on Apr. 2, 2025, the entire content of which is incorporated herein in its entirety.

### TECHNICAL FIELD

The present disclosure relates to the field of magnetic resonance technologies, and in particular, to a lumbar-vertebra magnetic resonance imaging method and apparatus, a computer device, a computer-readable storage medium, and a computer program product.

### BACKGROUND

With a rapid development of a magnetic resonance imaging technology, a multi-parameter quantitative imaging technology gains wide attention due to the capability of obtaining a plurality of quantitative imaging parameters to provide abundant information for clinical analysis. A main source of magnetic resonance imaging signals is hydrogen protons (i.e., H protons) in water, and since the lumbar vertebrae have a high fat content, signals generated by fat hydrogen protons in a magnetic resonance imaging process cannot be ignored, and a corresponding signal model is more complex than a single water hydrogen proton imaging model, so that water-fat separation is required when multi-parameter quantitative imaging is realized.

In the related art, water-fat separation imaging is performed mainly based on a low-resolution image, and for a high-resolution lumbar-vertebra imaging image, a water-fat separation process is easily subjected to interference by image noise, so that accurate water-fat separation is difficult to realize.

### SUMMARY

Based on this, in order to solve the above technical problem, there is provided a lumbar-vertebra magnetic resonance imaging method and apparatus, a computer device, a computer-readable storage medium, and a computer program product.

In a first aspect, the present disclosure provides a lumbar-vertebra magnetic resonance imaging method, including:
acquiring a sequence for lumbar vertebrae in a magnetic resonance imaging process; the sequence including water-fat in-phase echo signals corresponding to different echo times, and the water-fat in-phase echo signals being collected when hydrogen protons of water and fat are in a same phase;
determining an initial transverse relaxation rate map corresponding to the lumbar vertebrae and an initial field map representing inhomogeneity of a static magnetic field applied to the lumbar vertebrae according to a signal intensity of each water-fat in-phase echo signal, the echo time of each water-fat in-phase echo signal, and a pre-constructed signal model; the signal intensity of the water-fat in-phase echo signal in the signal model being a signal determined jointly by the transverse relaxation rate map and the field map of the inhomogeneity of the static magnetic field at the moment when the hydrogen protons of the water and the fat are in the same phase; and
taking the initial transverse relaxation rate map and the initial field map as initialization reconstruction parameters of water-fat separation, and reconstructing a water-fat separation image of the lumbar vertebrae.

In an embodiment, the water-fat in-phase echo signals include a plurality of water-fat in-phase echo signals obtained by a plurality of radio frequency pulse excitations, and the plurality of water-fat in-phase echo signals correspond to different flip angles; and
the method further includes:
acquiring a proton density map and a longitudinal relaxation time map of the lumbar vertebrae according to the initial transverse relaxation rate map, the flip angles corresponding to the multiple water-fat in-phase echo signals respectively, and a repetition time between the multiple radio frequency pulse excitations.

In an embodiment, the water-fat in-phase echo signal corresponds to a water-fat in-phase echo image with a first resolution; and
the flip angle corresponding to the water-fat in-phase echo signal is determined by the following steps:
acquiring an initial radio frequency field map corresponding to the radio frequency pulse excitation; a second resolution corresponding to the initial radio frequency field map being smaller than the first resolution;
adjusting the initial radio frequency field map with the second resolution to have the first resolution to obtain a target radio frequency field map; and
determining a flip angle of the radio frequency pulse excitation according to the target radio frequency field map as the flip angle corresponding to the water-fat in-phase echo signal.

In an embodiment, adjusting the initial radio frequency field map with the second resolution to have the first resolution includes:
performing linear interpolation on the initial radio frequency field map with the second resolution to obtain the initial radio frequency field map with the first resolution.

In an embodiment, the sequence further includes a plurality of water-fat out-of-phase echo signals, the water-fat out-of-phase echo signals being collected when the hydrogen protons of the water and the fat in the lumbar vertebrae are in different phases; and
taking the initial transverse relaxation rate map and the initial field map as the initialization reconstruction parameters of the water-fat separation, and reconstructing the water-fat separation image of the lumbar vertebrae includes:
taking the initial transverse relaxation rate map and the initial field map as the initialization reconstruction parameters for the water-fat separation;
performing water-fat separation image reconstruction according to the initialization reconstruction parameters, the water-fat out-of-phase echo signal, and the water-fat in-phase echo signal; and
obtaining a water map and a fat map of the lumbar vertebrae according to a reconstruction result, and determining a tissue fraction map of the lumbar vertebrae according to the water map and the fat map of the lumbar vertebrae.

In an embodiment, after the step of performing the water-fat separation image reconstruction according to the initialization reconstruction parameters, the water-fat out-of-phase echo signal, and the water-fat in-phase echo signal, the method further includes:
obtaining a target field map according to the reconstruction result; the target field map being obtained by iteratively updating the initial field map in the process of reconstructing the water-fat separation image; and
acquiring quantitative susceptibility mapping corresponding to the lumbar vertebrae according to the target field map.

In a second aspect, the present disclosure further provides a lumbar-vertebra magnetic resonance imaging apparatus, including:
a sequence acquiring module configured to acquire a sequence for lumbar vertebrae in a magnetic resonance imaging process; the sequence including water-fat in-phase echo signals corresponding to different echo times, and the water-fat in-phase echo signals being collected when hydrogen protons of water and fat are in a same phase;
an initial parameter estimating module configured to determine an initial transverse relaxation rate map corresponding to the lumbar vertebrae and an initial field map representing inhomogeneity of a static magnetic field applied to the lumbar vertebrae according to a signal intensity of each water-fat in-phase echo signal, the echo time of each water-fat in-phase echo signal, and a pre-constructed signal model; the signal intensity of the water-fat in-phase echo signal in the signal model being a signal determined jointly by the transverse relaxation rate map and the field map of the inhomogeneity of the static magnetic field at the moment when the hydrogen protons of the water and the fat are in the same phase; and
a water-fat separating module configured to take the initial transverse relaxation rate map and the initial field map as initialization reconstruction parameters of water-fat separation, and reconstruct a water-fat separation image of the lumbar vertebrae.

In a third aspect, the present disclosure further provides a computer device, including a memory and a processor, the memory storing a computer program, and the processor implementing the following steps when executing the computer program:
acquiring a sequence for lumbar vertebrae in a magnetic resonance imaging process; the sequence including water-fat in-phase echo signals corresponding to different echo times, and the water-fat in-phase echo signals being collected when hydrogen protons of water and fat are in a same phase;
determining an initial transverse relaxation rate map corresponding to the lumbar vertebrae and an initial field map representing inhomogeneity of a static magnetic field applied to the lumbar vertebrae according to a signal intensity of each water-fat in-phase echo signal, the echo time of each water-fat in-phase echo signal, and a pre-constructed signal model; the signal intensity of the water-fat in-phase echo signal in the signal model being a signal determined jointly by the transverse relaxation rate map and the field map of the inhomogeneity of the static magnetic field at the moment when the hydrogen protons of the water and the fat are in the same phase; and
taking the initial transverse relaxation rate map and the initial field map as initialization reconstruction parameters of water-fat separation, and reconstructing a water-fat separation image of the lumbar vertebrae.

In a fourth aspect, the present disclosure further provides a computer-readable storage medium having a computer program stored thereon, the computer program, when executed by a processor, implementing the following steps:
acquiring a sequence for lumbar vertebrae in a magnetic resonance imaging process; the sequence including water-fat in-phase echo signals corresponding to different echo times, and the water-fat in-phase echo signals being collected when hydrogen protons of water and fat are in a same phase;
determining an initial transverse relaxation rate map corresponding to the lumbar vertebrae and an initial field map representing inhomogeneity of a static magnetic field applied to the lumbar vertebrae according to a signal intensity of each water-fat in-phase echo signal, the echo time of each water-fat in-phase echo signal, and a pre-constructed signal model; the signal intensity of the water-fat in-phase echo signal in the signal model being a signal determined jointly by the transverse relaxation rate map and the field map of the inhomogeneity of the static magnetic field at the moment when the hydrogen protons of the water and the fat are in the same phase; and
taking the initial transverse relaxation rate map and the initial field map as initialization reconstruction parameters of water-fat separation, and reconstructing a water-fat separation image of the lumbar vertebrae.

In a fifth aspect, the present disclosure further provides a computer program product including a computer program which, when executed by a processor, implements the following steps:
acquiring a sequence for lumbar vertebrae in a magnetic resonance imaging process; the sequence including water-fat in-phase echo signals corresponding to different echo times, and the water-fat in-phase echo signals being collected when hydrogen protons of water and fat are in a same phase;
determining an initial transverse relaxation rate map corresponding to the lumbar vertebrae and an initial field map representing inhomogeneity of a static magnetic field applied to the lumbar vertebrae according to a signal intensity of each water-fat in-phase echo signal, the echo time of each water-fat in-phase echo signal, and a pre-constructed signal model; the signal intensity of the water-fat in-phase echo signal in the signal model being a signal determined jointly by the transverse relaxation rate map and the field map of the inhomogeneity of the static magnetic field at the moment when the hydrogen protons of the water and the fat are in the same phase; and
taking the initial transverse relaxation rate map and the initial field map as initialization reconstruction parameters of water-fat separation, and reconstructing a water-fat separation image of the lumbar vertebrae.

For the lumbar-vertebra magnetic resonance imaging method and apparatus, the computer device, the computer-readable storage medium, and the computer program product, the sequence for the lumbar vertebrae in the magnetic resonance imaging process may be acquired, the sequence including the water-fat in-phase echo signals corresponding to different echo times, and the water-fat in-phase echo signals being collected when the hydrogen protons of the water and the fat are in the same phase; then, the initial transverse relaxation rate map corresponding to the lumbar vertebrae and the initial field map representing the inhomogeneity of the static magnetic field applied to the lumbar vertebrae may be determined according to the signal intensity of each water-fat in-phase echo signal, the echo time of each water-fat in-phase echo signal, and the pre-constructed signal model, the signal intensity of the water-fat in-phase echo signal in the signal model being the signal determined jointly by the transverse relaxation rate map and the field map of the inhomogeneity of the static magnetic field at the moment when the hydrogen protons of the water and the fat are in the same phase; and then, the initial transverse relaxation rate map and the initial field map may be taken as the initialization reconstruction parameters of the water-fat separation, and the water-fat separation image of the lumbar vertebrae may be reconstructed. In this embodiment, by acquiring the water-fat in-phase echo signal of the lumbar vertebrae, the signals generated by the hydrogen protons of the water and the fat can be used as combined signals generated by the hydrogen protons of the same type under the condition of a high fat content in the lumbar vertebrae, so that the magnetic resonance imaging signal model for the lumbar vertebrae is effectively simplified, an accurate initial estimation result can be obtained for the field map and the transverse relaxation rate map based on the simplified signal model, and accuracy of the subsequent water-fat separation of the high-resolution lumbar vertebra image is effectively improved; and meanwhile, sufficient technical conditions are created for realizing multi-parameter quantitative imaging of the lumbar vertebrae.

### BRIEF DESCRIPTION OF THE DRAWINGS

In order to illustrate the technical solutions in the embodiments of the present disclosure or the related art more clearly, the drawings required for describing the embodiments of the present disclosure or the related art will be described briefly. Apparently, the following described drawings are merely for some embodiments of the present disclosure, and other related drawings can be derived from these drawings by those of ordinary skill in the art without any creative effort.

FIG. 1 is a schematic flow diagram of a lumbar-vertebra magnetic resonance imaging method according to an embodiment;

FIG. 2 is a schematic flow diagram of a step for determining a flip angle in an embodiment;

FIG. 3 is a schematic flow diagram of another lumbar-vertebra magnetic resonance imaging method according to an embodiment;

FIG. 4 is a circuit diagram of a high-resolution lumbar-vertebra multi-parameter quantitative imaging technology in an embodiment;

FIG. 5 is a structural block diagram of a lumbar-vertebra magnetic resonance imaging apparatus according to an embodiment;

FIG. 6 is an internal structure diagram of a computer device according to an embodiment; and

FIG. 7 is an internal structure diagram of another computer device according to an embodiment.

### DETAILED DESCRIPTION

The present disclosure will be described in further detail below with reference to the accompanying drawings and embodiments in order to make the objects, technical solutions, and advantages of the present disclosure more clear. It should be understood that the specific embodiments described herein are only for explaining the present disclosure, and not intended to limit the present disclosure.

In order to enable those skilled in the art to better understand the present disclosure, the related art will be described below.

With a rapid development of a magnetic resonance imaging technology, a multi-parameter quantitative imaging technology gains wide attention due to the capability of obtaining a plurality of quantitative imaging parameters to provide abundant information for clinical analysis. A used multi-parameter quantitative imaging model is complex, and signal models of different imaging sequences or imaging parts are different, so that the accurate and reliable quantitative imaging parameters can be obtained only by designing targeted algorithms for different imaging sequences and imaging parts.

A main source of magnetic resonance imaging signals is hydrogen protons (i.e., H protons) in water, and in the related art, fat contents in some tissue structures are quite low, and signals generated by hydrogen protons in fat can be ignored, thereby effectively simplifying the imaging signal model. For example, in a magnetic resonance craniocerebral multi-parameter quantitative imaging technology, the signals generated by the hydrogen protons of the fat can be ignored, thereby greatly simplifying the signal model. However, a fat content in lumbar vertebrae is high, the signals generated by the hydrogen protons of the fat cannot be ignored in a magnetic resonance imaging process, and since a precession frequency of the hydrogen protons in the fat is different from that of the hydrogen protons in the water, the signal model thereof is more complex than an imaging model of the hydrogen protons in the single water in the head, so that water-fat separation is required for realizing multi-parameter quantitative imaging.

In the related art, water-fat separation imaging is performed mainly based on a low-resolution image, and since a signal-to-noise ratio of the low-resolution image is high, the water-fat separation can be realized more accurately. However, in a high-resolution lumbar vertebra imaging image, the signal-to-noise ratio of the image is reduced, the water-fat separation process is easily subjected to interference by image noise, and accurate water-fat separation is difficult to realize.

Based on this, in order to solve the above technical problem, there is provided a lumbar-vertebra magnetic resonance imaging method and apparatus, a computer device, a computer-readable storage medium, and a computer program product.

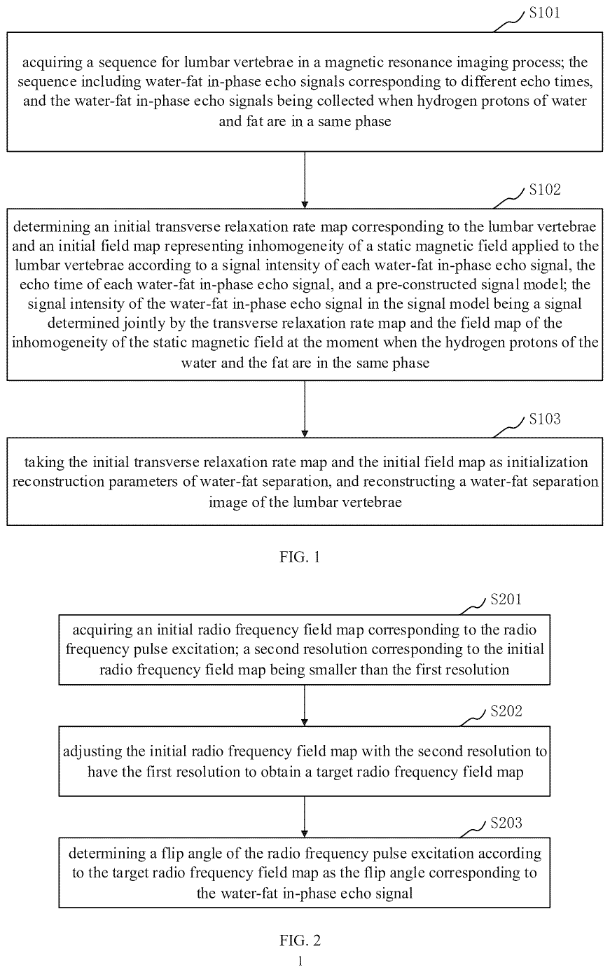

In an embodiment, as shown in FIG. 1, a lumbar-vertebra magnetic resonance imaging method is provided, and this embodiment is illustrated by applying the method to a terminal. It is to be understood that the method may alternatively be applied to a server or a system including a terminal and a server, and is implemented through interaction between the terminal and the server. In this embodiment, the method includes the following steps:
S101: acquiring a sequence for lumbar vertebrae in a magnetic resonance imaging process; the sequence including water-fat in-phase echo signals corresponding to different echo times, and the water-fat in-phase echo signals being collected when hydrogen protons of water and fat are in a same phase.

Specifically, properties of hydrogen protons within tissue structures in a magnetic field are used in magnetic resonance imaging. Under a static magnetic field environment, magnetic moments of the hydrogen protons are arranged along a magnetic field direction and are in a low energy state. When a radio frequency pulse is applied, energy of the radio frequency pulse is absorbed by the hydrogen protons, so that the magnetic moments of the hydrogen protons are deflected to generate a transverse magnetization vector, and in this case, the hydrogen protons are in an excited state. After the radio frequency pulse ceases, the hydrogen protons begin to revert from the excited state to a balance state, and the process is known as relaxation. During the relaxation, the transverse magnetization vector precesses around a main magnetic field direction and is gradually attenuated due to an interaction between the protons and a slight difference in the magnetic field environment. This attenuated transverse magnetization vector produces an induced current in a receiving coil, thereby producing an electromagnetic signal, i.e., an echo, that can be detected. By arranging a series of radio frequency pulse and/or gradient field pulse combinations according to a specified temporal order and parameter setting, a sequence including a plurality of echoes can be obtained.

Water and fat in tissue structures are two main hydrogen proton sources, hydrogen protons in the water and the fat have different precession frequencies in magnetic resonance imaging, and under the action of the specified magnetic field environment and a radio frequency pulse sequence, due to different chemical shifts of the hydrogen protons in the water and the fat, signal phases of the hydrogen protons in the water and the fat can change with time. When the hydrogen protons in the water and the fat are in the same phase, the echo signals collected in this case are the water-fat in-phase echo signals. For example, in a gradient echo sequence, by adjusting parameters such as a flip angle of the radio frequency pulse, and a strength and duration of a gradient field, the signals of the water and the fat can be in the same phase at a certain moment, and then, the water-fat in-phase echo signal is obtained.

In this step, the sequence collected for the lumbar vertebrae in the magnetic resonance imaging process may be acquired, the collected echo sequence may include the plurality of water-fat in-phase echo signals, and the plurality of water-fat in-phase echo signals correspond to different echo times. In some examples, the sequence may be a gradient echo sequence.
S102: determining an initial transverse relaxation rate map corresponding to the lumbar vertebrae and an initial field map representing inhomogeneity of a static magnetic field applied to the lumbar vertebrae according to a signal intensity of each water-fat in-phase echo signal, the echo time of each water-fat in-phase echo signal, and a pre-constructed signal model; the signal intensity of the water-fat in-phase echo signal in the signal model being a signal determined jointly by the transverse relaxation rate map and the field map of the inhomogeneity of the static magnetic field at the moment when the hydrogen protons of the water and the fat are in the same phase.

In practical applications, the corresponding signal model can be constructed in advance based on the water-fat in-phase echo signals. Specifically, at the collection moment of the water-fat in-phase echo signal, the signal phases of the signals generated by the hydrogen protons in the water and the hydrogen protons in the fat are the same, and based on this, the signal model can be simplified, the signals generated by the hydrogen protons in the water and the hydrogen protons in the fat are combined and regarded as a combined signal generated jointly by the hydrogen protons of the same type, and then, an association relationship between the signal intensity of the water-fat in-phase echo signal and the echo time, a transverse relaxation rate map, a field map of the static magnetic field, and the combined signal may be constructed to obtain the signal model, and in the signal model, the signal intensity of the water-fat in-phase echo signal can be determined jointly by the transverse relaxation rate map and the field map of the inhomogeneity of the static magnetic field at the moment when the hydrogen protons of the water and the fat are in the same phase. In an example, the signal model may be represented as follows:

$$S ( {TE}_{j} ) = ( {ρ}_{w} + {ρ}_{F} ) * {e}^{- i 2 π {f}_{s} {TE}_{j}} * {e}^{- {R}_{2}^{*} {TE}_{j}}$$

wherein S(TEⱼ) is the signal intensity of the jth water-fat in-phase echo signal, ρ_{w} is the signal generated by the hydrogen protons in the water, pp is the signal generated by the hydrogen protons in the fat, ρ_{w}+ρ_{F} is the combined signal generated jointly by the hydrogen protons in the water and the hydrogen protons in the fat, TEⱼ is the echo time of the jth water-fat in-phase echo signal,

$${R}_{2}^{*}$$

is the transverse relaxation rate diagram, and fₛ is the field map representing the inhomogeneity of the externally applied magnetic field.

Then, after the sequence is acquired, the transverse relaxation rate map for the lumbar vertebrae and the field map representing the inhomogeneity of the static magnetic field applied to the lumbar vertebrae can be calculated according to the signal intensity of the water-fat in-phase echo signals in the sequence, the echo time of each water-fat in-phase echo signal determined in the magnetic resonance imaging process and the pre-constructed signal model. For convenience of distinction, in this step, the transverse relaxation rate map and the field map determined according to the above signal model are referred to as the initial transverse relaxation rate map and the initial field map respectively.
S103: taking the initial transverse relaxation rate map and the initial field map as initialization reconstruction parameters of water-fat separation, and reconstructing a water-fat separation image of the lumbar vertebrae.

Specifically, in the magnetic resonance imaging, the water-fat separation refers to the process of obtaining a water map and a fat map by displaying the signals of the water and the fat respectively by using different properties of the hydrogen protons in the water and the fat.

In this step, after being estimated, the initial transverse relaxation rate map and the initial field map may be used as the initialization reconstruction parameters for the water-fat separation, wherein the initialization reconstruction parameters are parameters used as initialization information in an image iterative reconstruction process for the water-fat separation. Then, the initial transverse relaxation rate map and the initial field map are substituted into a related water-fat separation reconstruction algorithm, and the water-fat separation images of the lumbar vertebrae, i.e., the water map and the fat map of the lumbar vertebrae, can be reconstructed according to an iteration result of the algorithm.

In some exemplary embodiments, the water-fat separation image reconstruction may be performed based on any one of dual-echo out-of-phase imaging, a multi-echo Dixon technology, iterative decomposition water-fat imaging, or the like.

In the multi-echo Dixon technology, the plurality of echo signals at different echo times may be collected, and the plural echo signals are processed through a complex mathematical algorithm by utilizing a phase change rule of the water and fat signals at different echo times, thereby precisely separating the water and fat signals.

The iterative decomposition water-fat imaging is based on model driving, and water-fat separation is realized by establishing a water-fat signal model and decomposing collected magnetic resonance signals by using an iterative algorithm.

In the lumbar-vertebra magnetic resonance imaging method, the sequence for the lumbar vertebrae in the magnetic resonance imaging process may be acquired, the sequence including the water-fat in-phase echo signals corresponding to different echo times, and the water-fat in-phase echo signals being collected when the hydrogen protons of the water and the fat are in the same phase; then, the initial transverse relaxation rate map corresponding to the lumbar vertebrae and the initial field map representing the inhomogeneity of the static magnetic field applied to the lumbar vertebrae may be determined according to the signal intensity of each water-fat in-phase echo signal, the echo time of each water-fat in-phase echo signal, and the pre-constructed signal model, the signal intensity of the water-fat in-phase echo signal in the signal model being the signal determined jointly by the transverse relaxation rate map and the field map of the inhomogeneity of the static magnetic field at the moment when the hydrogen protons of the water and the fat are in the same phase; and then, the initial transverse relaxation rate map and the initial field map may be taken as the initialization reconstruction parameters of the water-fat separation, and the water-fat separation image of the lumbar vertebrae may be reconstructed. In this embodiment, by acquiring the water-fat in-phase echo signal of the lumbar vertebrae, the signals generated by the hydrogen protons of the water and the fat can be used as the combined signals generated by the hydrogen protons of the same type under the condition of the high fat content in the lumbar vertebrae, so that the magnetic resonance imaging signal model for the lumbar vertebrae is effectively simplified, an accurate initial estimation result (i.e., the initial transverse relaxation rate map and the initial field map) can be obtained for the field map and the transverse relaxation rate map based on the simplified signal model, and accuracy of the subsequent water-fat separation of the high-resolution lumbar vertebra image is effectively improved; and meanwhile, sufficient technical conditions are created for realizing multi-parameter quantitative imaging of the lumbar vertebrae.

In an exemplary embodiment, the water-fat in-phase echo signals include a plurality of water-fat in-phase echo signals obtained by a plurality of radio frequency pulse excitations, and the plurality of water-fat in-phase echo signals correspond to different flip angles. The flip angle is also called a toppling angle, and refers to an angle by which when the radio frequency pulse acts on the hydrogen protons in the tissue structure in the magnetic resonance imaging process, the magnetization vector of the hydrogen protons is flipped from an initial longitudinal direction (consistent with the main magnetic field direction) to a transverse plane. In some embodiments, two or more flip angles may be used, resulting in multiple water-fat in-phase echo signals. Exemplarily, when a signal-to-noise ratio in a water-fat in-phase echo is high (e.g., higher than a preset signal-to-noise ratio threshold), smaller flip angles (e.g., smaller than a preset angle threshold) may be used. For example, the two flip angles α1 and α2 are 3 degrees and 15 degrees respectively.

Correspondingly, the method may further include the following steps:
acquiring a proton density map and a longitudinal relaxation time map of the lumbar vertebrae according to the initial transverse relaxation rate map, the flip angles corresponding to the multiple water-fat in-phase echo signals respectively, and a repetition time between the multiple radio frequency pulse excitations.

Specifically, the repetition time (TR) between the multiple radio frequency pulse excitations may be determined. The repetition time is a time interval between two adjacent radio frequency pulse excitations, and may determine a collection cycle of the magnetic resonance signal, and after each radio frequency pulse excitation, the hydrogen protons in a tissue undergo the relaxation process, and the TR provides enough time for the hydrogen protons to relax to generate a detectable magnetic resonance signal at the next excitation. For example, in one pulse sequence, the repetition time is set to 1,000 ms, and then, the radio frequency pulse is applied every 1000 ms for the signal excitation.

In this embodiment, after the multiple water-fat in-phase echo signals are obtained and the flip angles of the water-fat in-phase echo signals are determined, the proton density map (PD) and the longitudinal relaxation time map (T1 relaxation map) of the lumbar vertebrae may be obtained according to the initial transverse relaxation rate map estimated in the step S102, the multiple water-fat in-phase echo signals, the flip angles corresponding to the multiple water-fat in-phase echo signals, and the repetition time between the multiple radio frequency pulse excitations.

In some exemplary embodiments, since the transverse relaxation rate map may reflect transverse relaxation properties of the tissue, the signals may be corrected in conjunction with the transverse relaxation rate map when the proton density map is obtained. For example, for a tissue with fast transverse relaxation, the signal thereof is attenuated fast, and if a proton density is judged only according to the echo signal intensity, an error is generated, and the signal attenuation can be compensated through the transverse relaxation rate map, so that the proton density can be reflected more accurately. After the water-fat in-phase echo signals under different flip angles are processed to remove influences of factors such as relaxation, an obtained signal intensity distribution can reflect a proton density distribution, and then, the proton density map is generated.

On the other hand, after the radio frequency pulses with different flip angles are applied, the longitudinal magnetization vector of the tissue can be flipped and restored to different degrees. In the plurality of water-fat in-phase echo signals with different flip angles, the collected signals contain information of the restoring process of the longitudinal magnetization vector. By analyzing a change of the signal intensity with time under different flip angles, a restoring curve of the longitudinal magnetization vector can be obtained.

For example, according to a basic theory of the magnetic resonance signal intensity, there is a mathematical relationship between the signal intensity S and the parameters such as the proton density PD, a longitudinal relaxation time T1, the flip angle α, and the repetition time TR, and the relationship can be expressed as S=PD×f(α,TR,T1), wherein f(α, TR,T1) is a function related to the flip angle, the repetition time, and the longitudinal relaxation time. The signal intensities S1 and S2 under the two different flip angles α1 and α2 are selected, S1=PD×f(α1,TR,T1) and S2=PD×f(α2,TR,T1) can be obtained according to formulas, the two equations are solved simultaneously to eliminate an influence of PD, so as to obtain an expression of T1, and a calculation is performed for each pixel to obtain the longitudinal relaxation time map. In an actual calculation, a more complex multi-flip-angle data fitting method can be adopted to improve calculation accuracy and stability.

In some exemplary embodiments, the PD map and a T1 quantitative image may be calculated according to the following formulas.

$$S ( {α}_{i} ) = \frac{S 0 * sin {α}_{i} * ( 1 - E 1 )}{1 - cos {α}_{i} * E 1}$$

$$E 1 = {e}^{- \frac{TR}{T 1}}$$

$$S 0 = PD * {e}^{- {R}_{2}^{*} TE}$$

wherein S(αᵢ) is the water-fat in-phase echo signal corresponding to the ith flip angle; αᵢ is the flip angle;

$${R}_{2}^{*}$$

is the initial transverse relaxation rate map; TR is the repetition time; and T1 is the longitudinal relaxation time.

In this embodiment, after the initial transverse relaxation rate map determined according to the water-fat in-phase echo is obtained, the proton density map and the longitudinal relaxation time map may be further quantitatively calculated in conjunction with the accurate and reliable initial transverse relaxation rate map, which is helpful in obtaining a reliable multi-parameter quantitative imaging result for the lumbar vertebrae.

In an exemplary embodiment, the water-fat in-phase echo signal may be presented by a water-fat in-phase echo image. For example, the water-fat in-phase echo image may be reconstructed from the water-fat in-phase echo signal, and for convenience of distinction, a resolution corresponding to the water-fat in-phase echo image is referred to as a first resolution.

As shown in FIG. 2, the flip angle corresponding to the water-fat in-phase echo signal may be determined by the following steps:
S201: acquiring an initial radio frequency field map corresponding to the radio frequency pulse excitation; a second resolution corresponding to the initial radio frequency field map being smaller than the first resolution.

In a specific implementation, when the proton density map and the longitudinal relaxation time map of the lumbar vertebrae are determined according to the water-fat in-phase echo signals under a plurality of flip angles, accuracy of the flip angles has a great influence on a parameter quantitative result. For example, the flip angle of the magnetization vector of the hydrogen proton is planned to be 3 degrees through a radio frequency, but the actual flip angle may be only 2.9 degrees, and if a subsequent calculation is performed directly according to the flip angle of 3 degrees, accuracy of the proton density map and the longitudinal relaxation time map is influenced.

In this regard, in this embodiment, a radio frequency field map B1 corresponding to the radio frequency pulse excitation may be obtained, and for convenience of distinction, the radio frequency field map B1 without resolution adjustment is referred to as the initial radio frequency field map, and meanwhile, a resolution corresponding to the initial radio frequency field map is referred to as the second resolution. In a specific implementation, the initial radio frequency field map can be obtained by fast scanning, and the second resolution of the initial radio frequency field map is smaller than the first resolution.
S202: adjusting the initial radio frequency field map with the second resolution to have the first resolution to obtain a target radio frequency field map.

After the initial radio frequency field map with the second resolution is obtained, the resolution adjustment may be performed on the initial radio frequency field map. That is, the initial radio frequency field map with the second resolution is adjusted to have the first resolution, and then, the radio frequency field map after the resolution adjustment may be referred to as the target radio frequency field map.
S203: determining a flip angle of the radio frequency pulse excitation according to the target radio frequency field map as the flip angle corresponding to the water-fat in-phase echo signal.

Since the radio frequency field map B1 contains information about an intensity and distribution of a radio frequency field, after the target radio frequency field map with the higher resolution is obtained, the flip angle of the radio frequency pulse excitation can be determined according to the target radio frequency field map, and the flip angle is used as the flip angle after the water-fat in-phase echo signal is corrected.

In this embodiment, the initial radio frequency field map with the second resolution is adjusted to have the first resolution to obtain the target radio frequency field map with the resolution matched with that of a water-fat in-phase echo image sequence, so that the flip angle can be corrected based on the target radio frequency field map with matched pixel precision, and precision of the proton density map and the longitudinal relaxation time map subsequently determined according to each flip angle is effectively improved.

In an exemplary embodiment, in the step S202, adjusting the initial radio frequency field map with the second resolution to have the first resolution to obtain the target radio frequency field map may include the following steps: performing linear interpolation on the initial radio frequency field map with the second resolution to obtain the initial radio frequency field map with the first resolution.

In practical applications, the initial radio frequency field map with the same resolution as the water-fat in-phase echo image sequence can be obtained by performing multi-linear interpolation on the initial radio frequency field map with the second resolution, thus effectively improving an adjustment speed of the resolution.

In an exemplary embodiment, the sequence further includes a plurality of water-fat out-of-phase echo signals, and the water-fat out-of-phase echo signals are signals collected when the hydrogen protons of the water and the fat in the lumbar vertebrae are in different phases, for example, collected when the phases of the hydrogen protons of the water and the fat are different by x (x is greater than 0) degrees; correspondingly, in the step S103, taking the initial transverse relaxation rate map and the initial field map as the initialization reconstruction parameters of the water-fat separation, and reconstructing the water-fat separation image of the lumbar vertebrae may include the following steps:
taking the initial transverse relaxation rate map and the initial field map as the initialization reconstruction parameters for the water-fat separation; performing water-fat separation image reconstruction according to the initialization reconstruction parameters, the water-fat out-of-phase echo signal, and the water-fat in-phase echo signal; and obtaining a water map and a fat map of the lumbar vertebrae according to a reconstruction result, and determining a tissue fraction map of the lumbar vertebrae according to the water map and the fat map of the lumbar vertebrae.

Specifically, due to different chemical environments, the hydrogen protons in the water and the fat have different precession frequencies, the difference is called a chemical shift, and under a certain magnetic field intensity, the hydrogen protons in the fat have a slightly slower precession frequency than the hydrogen protons in the water. During the magnetic resonance imaging, this frequency difference causes the phases of the signals of the water and the fat to change with time. At some specific time points, the phases of the signals of the water and the fat are the same, and the signals of the water and the fat are mutually superposed in the collected echo signals; and at other time points, the phases of the signals of the water and the fat are different (e.g., opposite), and the signals of the water and the fat are counteracted in the collected echo signals. The water-fat separation can be realized by collecting and processing the signals with different phases.

After being obtained, the initial transverse relaxation rate map and the initial field map can be used as the initialization reconstruction parameters for the water-fat separation, and the water-fat separation image reconstruction is performed in conjunction with the water-fat out-of-phase echo signal and the water-fat in-phase echo signal. In some exemplary embodiments, the water-fat separation image reconstruction may be performed based on the dual-echo out-of-phase imaging technology.

In the double-echo out-of-phase imaging, the gradient echo sequence is adopted, and after each radio frequency pulse excitation, two echo signals are respectively collected at water-fat in-phase and out-of-phase time points to obtain the water-fat in-phase echo and a water-fat out-of-phase echo, and in the water-fat in-phase echo, the water and fat signals are added; and in the water-fat out-of-phase echo, the water and fat signals are subtracted. By performing mathematical operations on the two echo signals, such as addition and subtraction of the intensity of the in-phase image signal and the intensity of the out-of-phase image signal, the maps of the water and the fat can be respectively obtained to realize the water-fat separation.

In the process of water-fat separation image reconstruction, the initialization reconstruction parameters can be used as initial conditions, the water map and the fat map are continuously updated iteratively in conjunction with a relevant iterative algorithm, and when an iterative termination condition is met, the water map and the fat map containing the lumbar vertebrae can be obtained, and then, the tissue fraction map of the lumbar vertebrae is determined according to the water map and the fat map of the lumbar vertebrae, and then, the tissue fraction map can be used as a water-fat separation result.

In some exemplary embodiments, the water map and the fat map can be directly used as the water-fat separation result of the lumbar vertebrae; in some other embodiments, the tissue fraction map such as a proton density fat fraction map (PDFF) may alternatively be generated from the water map and the fat map in the reconstruction result, and the water-fat separation result may be obtained from the water map, the fat map, and the proton density fat fraction map. The proton density fat fraction map can reflect an image of a relative proportion of the fat content in the tissue structure, and a percentage of the fat in the tissue relative to the total proton density is presented in the form of an image by analyzing and calculating components of the fat and the water in the magnetic resonance signal.

In this embodiment, the initial transverse relaxation rate map and the initial field map are used as the initialization reconstruction parameters for the water-fat separation, and then, the water map and the fat map of the lumbar vertebrae are iteratively reconstructed in conjunction with the water-fat out-of-phase echo signal, so that accurate initialization of the water-fat separation iterative reconstruction can be realized on the basis of an accurate and reliable initial parameter estimation result, trapping in a local extreme value in the water-fat separation process is effectively prevented, a local optimal solution is avoided, and a necessary guarantee is provided for precisely realizing multi-parameter quantification.

In an exemplary embodiment, after the step of performing the water-fat separation image reconstruction according to the initialization reconstruction parameters, the water-fat out-of-phase echo signal, and the water-fat in-phase echo signal, the method may further include the following steps:
obtaining a target field map according to the reconstruction result; the target field map being obtained by iteratively updating the initial field map in the process of reconstructing the water-fat separation image; and acquiring quantitative susceptibility mapping corresponding to the lumbar vertebrae according to the target field map.

In a specific implementation, during the water-fat separation image reconstruction, the water map and the fat map are continuously updated iteratively, the initial field map fₛ and the initial transverse relaxation rate map R2* can be updated iteratively, and when the iterative termination condition is met and the reconstruction result is obtained, the updated target field map can be obtained. Compared with the initial field map, accuracy of the target field map can be effectively improved in conjunction with the elaborately iterative updating of the water-fat out-of-phase echo signal and the water-fat in-phase echo signal in the water-fat separation process.

Subsequently, the quantitative susceptibility mapping (QSM) corresponding to the lumbar vertebrae can be obtained according to the target field map. For example, after the target field map is subjected to background removal, dipole deconvolution, or the like, the quantitative susceptibility mapping can be finally obtained.

In this embodiment, accuracy of the lumbar-vertebra multi-parameter quantitative imaging result can be further improved by acquiring the target field map obtained in the water-fat separation image reconstruction process and generating the quantitative susceptibility mapping according to the target field map.

In order to enable those skilled in the art to better understand the above steps, the embodiments of the present disclosure are exemplarily illustrated below with an example, but it should be understood that the embodiments of the present disclosure are not limited thereto.

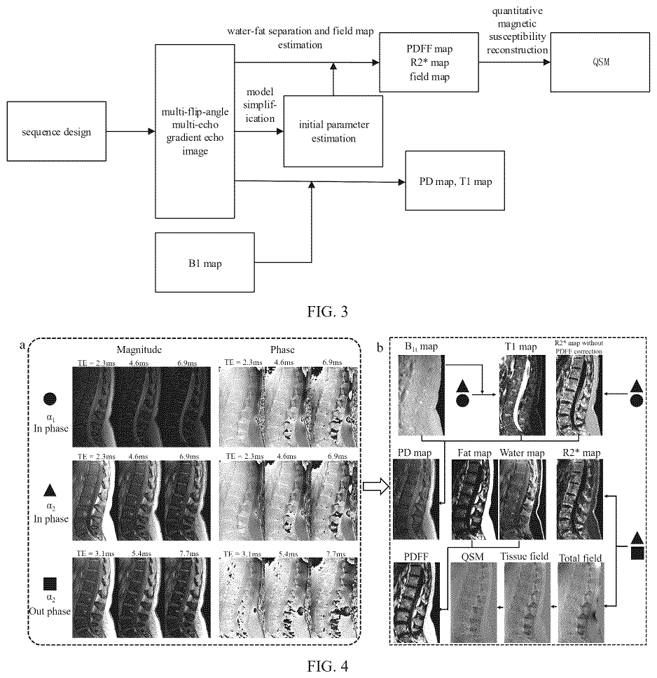

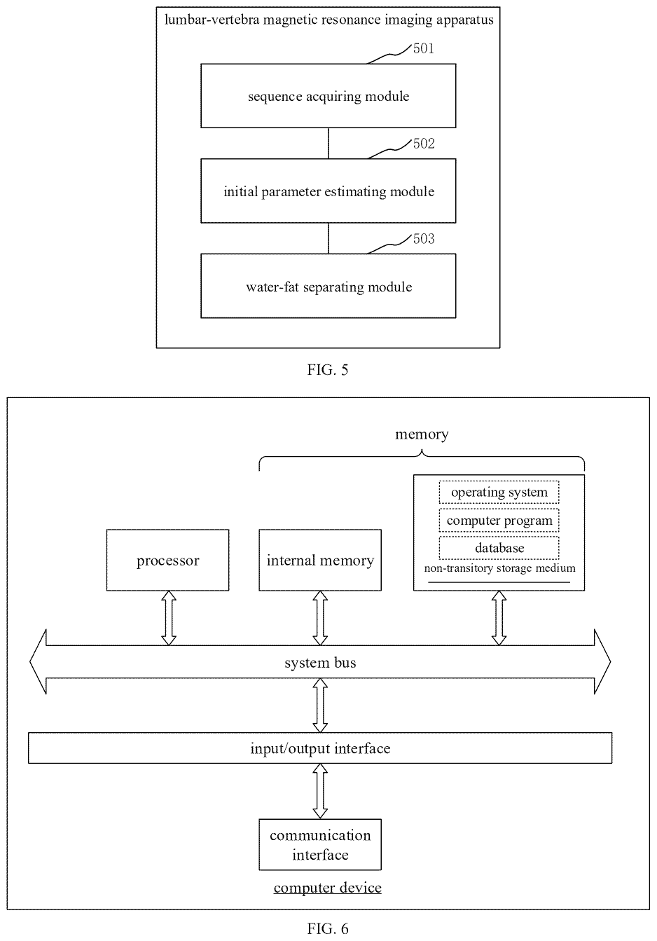

As shown in FIG. 3, a sequence design may be performed first to acquire a multi-flip-angle multi-echo gradient echo image. In the sequence design process, a multi-flip-angle design and an in-phase echo design are involved. Taking FIG. 4 as an example, water-fat in-phase echo signals at two flip angles (i.e., α1 and α2) may be collected and include the water-fat in-phase echo signal at the flip angle α1 (which may be represented by •) and the water-fat in-phase echo signal at the flip angle α2 (which may be represented by ▴), wherein the water-fat in-phase echo signals at the flip angles α1 and α2 may be collected by determining that hydrogen protons of water and fat are in the same phase when echo times are 2.3 ms, 4.6 ms, and 6.9 ms. In addition, a water-fat out-of-phase echo signal (which can be represented by ▪) at the flip angle α2 can be obtained and may be collected by determining that the hydrogen protons of the water and the fat are in different phases (for example, opposite phases) when the echo times are 3.1 ms, 5.4 ms, and 7.7 ms. The water-fat in-phase echo signal and the water-fat out-of-phase echo signal mentioned above are presented in the form of echo images.

Thus, a multi-flip-angle multi-echo gradient echo image can be obtained, wherein the multi-flip-angle multi-echo gradient echo image can be a high-resolution image, and for example, a resolution of the multi-flip-angle multi-echo gradient echo image can be higher than a preset threshold. Subsequently, field map estimation, water-fat separation and quantitative calculations of a PD map and a T1 map can be carried out in conjunction with a multi-flip-angle multi-echo gradient echo image to obtain a plurality of accurate and stable quantitative parameters.

Specifically, in the field map estimation, an initialized

$${R}_{2}^{*}$$

map (“R2* map without PDFF correction” in FIG. 4) and a map fₛ may be determined based on the water-fat in-phase echo signal at at least one flip angle and a simplified signal model. In some embodiments, the initialized

$${R}_{2}^{*}$$

and fₛ may be determined according to the water-fat in-phase echo signal under according to the water-fat in-phase echo signal under one flip angle and the simplified signal model, and in this case, image signal-to-noise ratios of the water-fat in-phase echo signals corresponding to multiple flip angles may be compared, the water-fat in-phase echo signal with a higher signal-to-noise ratio is selected to obtain an initial parameter estimation result, and water-fat separation difficulty is reduced. For example, if α1 and α2 are 3 degrees and 15 degrees respectively, an image obtained under α2 has a higher signal-to-noise ratio than an image obtained under al, and in this case, the initialized

$${R}_{2}^{*}$$

and fₛ may be determined by using the water-fat in-phase echo signal obtained under α2. In some other embodiments, the initialized

$${R}_{2}^{*}$$

and fₛ may be determined simultaneously in conjunction with the water-fat in-phase echo signals at multiple flip angles and the simplified signal model. For example, for the water-fat in-phase echo signal under each flip angle, the initialized R2 and fₛ corresponding to the flip angle can be determined according to the sequence and the signal model; and then, final initial parameter estimation results

$${R}_{2}^{*}$$

and fₛ are determined according to the

$${R}_{2}^{*}$$

and fₛ corresponding to the plurality of flip angles.

Subsequently, a B1 map (i.e., Bₗₜ map in FIG. 4) of a radio frequency field may be acquired, a resolution of the B1 map is adjusted to be the same as that of the multi-flip-angle multi-echo gradient echo image by multi-linear interpolation, and a PD image and a T1 image are calculated from the B1 map,

$${R}_{2}^{*}$$

in the initial parameter estimation result, the water-fat in-phase echo signal at the flip angle α1 and the water-fat in-phase echo signal at the flip angle α2 (i.e., a multi-flip-angle echo sequence in the sequence design).

On the other hand, during water-fat separation, water-fat separation image reconstruction may be performed by the initial parameter estimation results

$${R}_{2}^{*}$$

and fₛ in conjunction with the water-fat out-of-phase echo signal and the water-fat in-phase echo signal at the flip angle of α2 (i.e., the multi-echo gradient echo image in the sequence design), and a final water map (the “water map” in FIG. 4), a final fat map (the “fat map” in FIG. 4),

$${R}_{2}^{*}$$

(the “R2* map” in FIG. 4), and a target field map fₛ (the “total field” in FIG. 4) are obtained through iterative reconstruction. For the target field map fₛ, QSM can be finally obtained after background removal and dipole deconvolution.

It should be understood that, although the steps in the flow charts involved in the above embodiments are shown in sequence as indicated by the arrows, the steps are not necessarily performed in sequence as indicated by the arrows. Unless explicitly stated herein, the steps are not limited to being performed in the exact order and may be performed in other orders. At least part of the steps in the flow charts involved in the above embodiments may include multiple steps or multiple stages, which are not necessarily performed at the same moment, but may be performed at different moments, and the steps or the stages are not necessarily performed in sequence, but may be performed alternately with other steps or at least part of the steps or the stages in other steps.

Based on the same inventive concept, the embodiment of the present disclosure further provides a lumbar-vertebra magnetic resonance imaging apparatus for implementing the above lumbar-vertebra magnetic resonance imaging method. The implementation solution for solving problems provided by the apparatus is similar to the implementation solution described in the above method, and therefore, for the specific definitions in one or more embodiments of the lumbar-vertebra magnetic resonance imaging apparatus provided below, reference can be made to the definitions of the lumbar-vertebra magnetic resonance imaging method in the above description, and the definitions are not repeated herein.

In an exemplary embodiment, as shown in FIG. 5, there is provided a lumbar-vertebra magnetic resonance imaging apparatus, including:
a sequence acquiring module 501 configured to acquire a sequence for lumbar vertebrae in a magnetic resonance imaging process; the sequence including water-fat in-phase echo signals corresponding to different echo times, and the water-fat in-phase echo signals being collected when hydrogen protons of water and fat are in a same phase;
an initial parameter estimating module 502 configured to determine an initial transverse relaxation rate map corresponding to the lumbar vertebrae and an initial field map representing inhomogeneity of a static magnetic field applied to the lumbar vertebrae according to a signal intensity of each water-fat in-phase echo signal, the echo time of each water-fat in-phase echo signal, and a pre-constructed signal model; the signal intensity of the water-fat in-phase echo signal in the signal model being a signal determined jointly by the transverse relaxation rate map and the field map of the inhomogeneity of the static magnetic field at the moment when the hydrogen protons of the water and the fat are in the same phase; and
a water-fat separating module 503 configured to take the initial transverse relaxation rate map and the initial field map as initialization reconstruction parameters of water-fat separation, and reconstruct a water-fat separation image of the lumbar vertebrae.

In an embodiment, the water-fat in-phase echo signals include a plurality of water-fat in-phase echo signals obtained by a plurality of radio frequency pulse excitations, and the plurality of water-fat in-phase echo signals correspond to different flip angles; and
The apparatus further includes:
a first quantitative parameter calculating module configured to acquire a proton density map and a longitudinal relaxation time map of the lumbar vertebrae according to the initial transverse relaxation rate map, the flip angles corresponding to the multiple water-fat in-phase echo signals respectively, and a repetition time between the multiple radio frequency pulse excitations.

In an embodiment, the water-fat in-phase echo signal corresponds to a water-fat in-phase echo image with a first resolution; and
the first quantitative parameter calculating module is further configured to:
acquire an initial radio frequency field map corresponding to the radio frequency pulse excitation; a second resolution corresponding to the initial radio frequency field map being smaller than the first resolution;
adjust the initial radio frequency field map with the second resolution to have the first resolution to obtain a target radio frequency field map; and
determine a flip angle of the radio frequency pulse excitation according to the target radio frequency field map as the flip angle corresponding to the water-fat in-phase echo signal.

In an embodiment, the first quantitative parameter calculating module is further configured to:
perform linear interpolation on the initial radio frequency field map with the second resolution to obtain the initial radio frequency field map with the first resolution.

In an embodiment, the sequence further includes a plurality of water-fat out-of-phase echo signals, the water-fat out-of-phase echo signals being collected when the hydrogen protons of the water and the fat in the lumbar vertebrae are in different phases; and
The water-fat separating module 503 is configured to:
take the initial transverse relaxation rate map and the initial field map as the initialization reconstruction parameters for the water-fat separation;
perform water-fat separation image reconstruction according to the initialization reconstruction parameters, the water-fat out-of-phase echo signal, and the water-fat in-phase echo signal; and
obtain a water map and a fat map of the lumbar vertebrae according to a reconstruction result, and determine a tissue fraction map of the lumbar vertebrae according to the water map and the fat map of the lumbar vertebrae.

In an embodiment, the apparatus further includes a second quantitative parameter calculating module, the second quantitative parameter calculating module being configured to:
obtain a target field map according to the reconstruction result; the target field map being obtained by iteratively updating the initial field map in the process of reconstructing the water-fat separation image; and
acquire quantitative susceptibility mapping corresponding to the lumbar vertebrae according to the target field map.

The modules in the lumbar-vertebra magnetic resonance imaging apparatus may be wholly or partially implemented by software, hardware and a combination thereof. The modules may be embedded in or independent of the processor in the computer device in hardware, or may be stored in the memory in the computer device in software, such that the processor can conveniently call the modules to execute the operations corresponding to the modules.

In an exemplary embodiment, there is provided a computer device, which may be a server, an internal structure diagram of which may be shown in FIG. 6. The computer device includes a processor, a memory, an input/output (I/O) interface, and a communication interface. The processor, the memory, and the input/output interface are connected through a system bus, and the communication interface is connected to the system bus through the input/output interface. The processor of the computer device is configured to provide computing and control capabilities. The memory of the computer device includes a non-transitory storage medium and an internal memory. The non-transitory storage medium stores an operating system, a computer program, and a database. The internal memory provides an environment for running of the operating system and the computer program in the non-transitory storage medium. The database of the computer device is configured to store magnetic resonance imaging data including a lumbar-vertebra echo sequence. The input/output interface of the computer device is configured to exchange information between the processor and an external device. The communication interface of the computer device is configured to be communicated with an external terminal through a network connection. The computer program is executed by the processor to implement a lumbar-vertebra magnetic resonance imaging method.

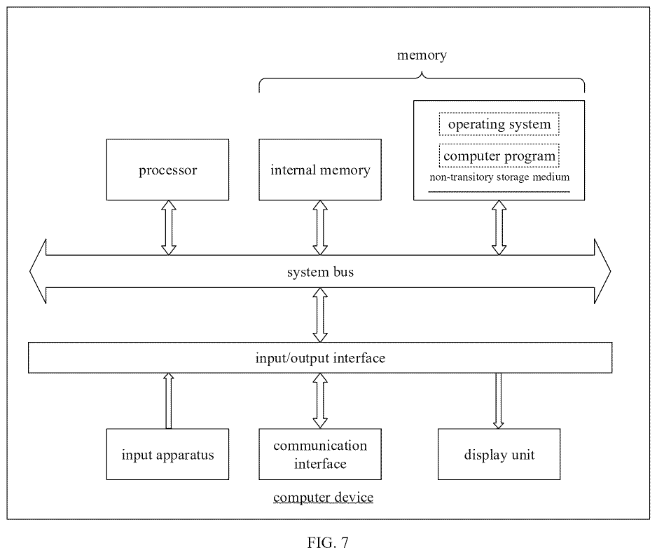

In an exemplary embodiment, there is provided a computer device, which may be a terminal, an internal structure diagram of which may be shown in FIG. 7. The computer device includes a processor, a memory, an input/output interface, a communication interface, a display unit, and an input apparatus. The processor, the memory, and the input/output interface are connected through a system bus, and the communication interface, the display unit, and the input apparatus are connected to the system bus through the input/output interface. The processor of the computer device is configured to provide computing and control capabilities. The memory of the computer device includes a non-transitory storage medium and an internal memory. The non-transitory storage medium stores an operating system and a computer program. The internal memory provides an environment for running of the operating system and the computer program in the non-transitory storage medium. The input/output interface of the computer device is configured to exchange information between the processor and an external device. The communication interface of the computer device is configured to be communicated with an external terminal in a wired or wireless mode, and the wireless mode can be implemented through WIFI, a mobile cellular network, near field communication (NFC), or other technologies. The computer program is executed by the processor to implement a lumbar-vertebra magnetic resonance imaging method. The display unit of the computer device is configured to form a visually visible picture and can be a display screen, a projection apparatus, or a virtual reality imaging apparatus. The display screen may be a liquid crystal display screen or an electronic ink display screen, and the input apparatus of the computer device may be a touch layer covering the display screen, or may be a key, a trackball or a touch pad arranged on a casing of the computer device, or may be an external keyboard, touch pad, mouse, or the like.

Those skilled in the art will appreciate that the structure shown in FIG. 6 and FIG. 7 is only a block diagram of a part of the structure associated with the application solution and does not constitute a limitation on the computer device to which the application solution is applied, and a particular computer device may include more or fewer components than shown components, or combine certain components, or have a different arrangement of components.

In an embodiment, there is provided a computer device, including a memory and a processor, the memory storing a computer program, and the processor implementing the steps of the above method embodiments when executing the computer program.

In an embodiment, there is provided a computer-readable storage medium having a computer program stored thereon, the computer program, when executed by a processor, implementing the steps of the above method embodiments.

In an embodiment, there is provided a computer program product including a computer program which, when executed by a processor, implements the steps in the above method embodiments.

It should be noted that user information (including but not limited to user device information, user personal information, or the like) and data (including but not limited to data for analysis, stored data, displayed data, or the like) involved in the present disclosure are information and data authorized by the user or sufficiently authorized by each party, and collection, use and processing of relevant data require compliance with relevant regulations.

It will be understood by those skilled in the art that all or part of the processes of the method according to the embodiments described above may be implemented by a computer program instructing related hardware, and the computer program may be stored in a non-transitory computer-readable storage medium, and when executed, may include the processes of the embodiments of the method described above. Any reference to memories, databases or other media used in the embodiments of the present disclosure can include at least one of a non-transitory memory and a transitory memory. The non-transitory memory may include a read-only memory (ROM), a magnetic tape, a floppy disk, a flash memory, an optical memory, a high-density embedded non-transitory memory, a resistive random access memory (ReRAM), a magnetoresistive random access memory (MRAM), a ferroelectric random access memory (FRAM), a phase change memory (PCM), a graphene memory, or the like. The transitory memory can include a random access memory (RAM), an external cache memory, or the like. By way of illustration and not limitation, the RAM can take many forms, such as a static random access memory (SRAM), a dynamic random access memory (DRAM), or the like. The databases involved in the embodiments of the present disclosure may include at least one of relational and non-relational databases. The non-relational database may include, but is not limited to, a block chain-based distributed database, or the like. The processors referred to in the embodiments of the present disclosure may include, but are not limited to, general processors, central processors, graphics processors, digital signal processors, programmable logic units, data processing logic units based on quantum calculations, artificial intelligence (AI) processors, or the like.

The technical features of the above-mentioned embodiments can be combined arbitrarily. In order to make the description concise, not all possible combinations of the technical features are described in the embodiments. However, as long as there is no contradiction in the combination of these technical features, the combinations should be considered as in the scope of the application.

The above-described embodiments are only several implementations of the present disclosure, and the descriptions are relatively specific and detailed, but they should not be construed as limiting the scope of the present disclosure. It should be understood by those of ordinary skill in the art that various modifications and improvements can be made without departing from the concept of the present disclosure, and all fall within the protection scope of the present disclosure. Therefore, the protection scope of the present disclosure should be subject to the appended claims.

1. A lumbar-vertebra magnetic resonance imaging method, comprising:
acquiring a sequence for lumbar vertebrae in a magnetic resonance imaging process; the sequence comprising water-fat in-phase echo signals corresponding to different echo times, and the water-fat in-phase echo signals being collected when hydrogen protons of water and fat are in a same phase;
determining an initial transverse relaxation rate map corresponding to the lumbar vertebrae and an initial field map representing inhomogeneity of a static magnetic field applied to the lumbar vertebrae according to a signal intensity of each water-fat in-phase echo signal, the echo time of each water-fat in-phase echo signal, and a pre-constructed signal model; the signal intensity of the water-fat in-phase echo signal in the signal model being a signal determined jointly by the transverse relaxation rate map and the field map of the inhomogeneity of the static magnetic field at the moment when the hydrogen protons of the water and the fat are in the same phase; and
taking the initial transverse relaxation rate map and the initial field map as initialization reconstruction parameters of water-fat separation, and reconstructing a water-fat separation image of the lumbar vertebrae.

2. The method according to claim 1, wherein the water-fat in-phase echo signals comprise a plurality of water-fat in-phase echo signals obtained by a plurality of radio frequency pulse excitations, and the plurality of water-fat in-phase echo signals correspond to different flip angles; and
the method further comprises:
acquiring a proton density map and a longitudinal relaxation time map of the lumbar vertebrae according to the initial transverse relaxation rate map, the flip angles corresponding to the multiple water-fat in-phase echo signals respectively, and a repetition time between the multiple radio frequency pulse excitations.

3. The method according to claim 2, wherein the water-fat in-phase echo signal corresponds to a water-fat in-phase echo image with a first resolution; and
the flip angle corresponding to the water-fat in-phase echo signal is determined by the following steps:
acquiring an initial radio frequency field map corresponding to the radio frequency pulse excitation; a second resolution corresponding to the initial radio frequency field map being smaller than the first resolution;
adjusting the initial radio frequency field map with the second resolution to have the first resolution to obtain a target radio frequency field map; and
determining a flip angle of the radio frequency pulse excitation according to the target radio frequency field map as the flip angle corresponding to the water-fat in-phase echo signal.

4. The method according to claim 3, wherein adjusting the initial radio frequency field map with the second resolution to have the first resolution comprises:
performing linear interpolation on the initial radio frequency field map with the second resolution to obtain the initial radio frequency field map with the first resolution.

5. The method according to claim 1, wherein the sequence further comprises a plurality of water-fat out-of-phase echo signals, the water-fat out-of-phase echo signals being collected when the hydrogen protons of the water and the fat in the lumbar vertebrae are in different phases; and
taking the initial transverse relaxation rate map and the initial field map as the initialization reconstruction parameters of the water-fat separation, and reconstructing the water-fat separation image of the lumbar vertebrae comprises:
taking the initial transverse relaxation rate map and the initial field map as the initialization reconstruction parameters for the water-fat separation;
performing water-fat separation image reconstruction according to the initialization reconstruction parameters, the water-fat out-of-phase echo signal, and the water-fat in-phase echo signal; and
obtaining a water map and a fat map of the lumbar vertebrae according to a reconstruction result, and determining a tissue fraction map of the lumbar vertebrae according to the water map and the fat map of the lumbar vertebrae.

6. The method according to claim 5, wherein after the step of performing the water-fat separation image reconstruction according to the initialization reconstruction parameters, the water-fat out-of-phase echo signal, and the water-fat in-phase echo signal, the method further comprises:
obtaining a target field map according to the reconstruction result; the target field map being obtained by iteratively updating the initial field map in the process of reconstructing the water-fat separation image; and
acquiring quantitative susceptibility mapping corresponding to the lumbar vertebrae according to the target field map.

7. A lumbar-vertebra magnetic resonance imaging apparatus, comprising:
a sequence acquiring module configured to acquire a sequence for lumbar vertebrae in a magnetic resonance imaging process; the sequence comprising water-fat in-phase echo signals corresponding to different echo times, and the water-fat in-phase echo signals being collected when hydrogen protons of water and fat are in a same phase;
an initial parameter estimating module configured to determine an initial transverse relaxation rate map corresponding to the lumbar vertebrae and an initial field map representing inhomogeneity of a static magnetic field applied to the lumbar vertebrae according to a signal intensity of each water-fat in-phase echo signal, the echo time of each water-fat in-phase echo signal, and a pre-constructed signal model; the signal intensity of the water-fat in-phase echo signal in the signal model being a signal determined jointly by the transverse relaxation rate map and the field map of the inhomogeneity of the static magnetic field at the moment when the hydrogen protons of the water and the fat are in the same phase; and
a water-fat separating module configured to take the initial transverse relaxation rate map and the initial field map as initialization reconstruction parameters of water-fat separation, and reconstruct a water-fat separation image of the lumbar vertebrae.

8. A computer device, comprising a memory and a processor, the memory storing a computer program, and the processor implementing the steps of the method according to claim 1 when executing the computer program.

9. A computer-readable storage medium having a computer program stored thereon, the computer program, when executed by a processor, implementing the steps of the method according to claim 1.

10. A computer program product comprising a computer program, the computer program, when executed by a processor, implementing the steps of the method according to claim 1.
